(19) United States
(12) Patent Application Publication
(10) Pub. No.: US 2018/0317822 A1
(43) Pub. Date: Nov. 8, 2018

(54) NONINVASIVE OPTICAL IN-VIVO DETERMINING OF GLUCOSE CONCENTRATION IN FLOWING BLOOD

(71) Applicant: NIRLUS Engineering AG, Lübeck (DE)

(72) Inventor: Vera HERRMANN, Luebeck (DE)

(21) Appl. No.: 15/774,927

(22) PCT Filed: Nov. 16, 2016
(86) PCT No.: PCT/EP2016/077833

§ 371 Date: May 9, 2018

(30) Foreign Application Priority Data
Nov. 20, 2015 (EP) 15195694.3

(51) Int. Cl. A61B 5/145; A61B 5/1455; A61B 5/00; A61B 8/08

(52) U.S. Cl. CPC A61B 5/14532; A61B 8/085; A61B 5/7228; A61B 5/1455

(57) ABSTRACT

The invention relates to a method and a device for the non-invasive optical in-vivo determining of the glucose concentration in flowing blood in a blood vessel inside a body, wherein the body is irradiated with ultrasonic radiation with an ultrasonic frequency to mark a blood vessel, wherein the body with the blood vessel is illuminated with light having at least one first light wavelength, wherein the intensity of the back-scattered light depends on the glucose concentration, wherein the body with the blood vessel is illuminated with light having a second light wavelength that lies in the range of a water absorption line, the position of which depends on the temperature of the blood, wherein the respective back-scattered light is detected by at least one detector, wherein, using an evaluation unit, respective signal portions modulated by a modulation frequency depending on the ultrasonic frequency are extracted from the detector signals measured at the detector, wherein an indicator value for the glucose concentration is determined from the signal portion determined at the first wavelength, wherein the indicator value is corrected by the signal portion of the second light wavelength for the compensation of the temperature dependency.

The invention relates to a method of the noninvasive in vivo determination of the glucose concentration in flowing blood in a blood vessel inside a body. The invention is primarily concerned with optical analysis using light, particularly by laser radiation through analysis of the backscattered light, with the location of the measurement, namely the blood vessel, being “marked” by (pulsed) ultrasonic radiation. In the context of the invention, the aim is to determine the blood glucose concentration, or blood sugar level, in vivo, that is without direct contact with the blood, thus eliminating the need to draw of blood. For example, diabetics need to quickly and easily measure using a compact and portable measuring apparatus, for example, that rapidly provides reliable values through contact with the skin at most and without injuring the skin.

One method of optically measuring characteristics of flowing blood with ultrasound localization is known for example from EP 1 601 285 [U.S. Pat. No. 7,251,518]. The ultrasonic radiation is focused inside a central blood vessel and a fixed pulse length and repetition time for the ultrasonic radiation is predetermined. Moreover, a light source and an adjacent sensor for detecting the light backscattered to the skin surface is positioned over the blood vessel such that the spacing between light source and the majority of the light receptors of the sensor corresponds to the depth of the blood tissue being examined. The target tissue is irradiated with at least two discrete light wavelengths, and the backscattered light is measured and integrated via the sensor surface and a plurality of ultrasonic pulses. By interacting with blood and tissue, the ultrasonic wave field alters the optical characteristics, particularly the reflectance and scattering power. This results in a modulation of the backscattered light at the frequency of the ultrasonic radiation, enabling the modulated portion to be extracted during the analysis.

In connection with the determination of the blood glucose concentration, DE 10 2006 036 920 [U.S. Pat. No. 8,391,939] describes a method of the spectrometric determination of the blood glucose concentration in pulsatingly flowing blood, using a wavelength in the range from 1560 to 1630 nm, preferably 1600 nm. Moreover, a second wavelength in the wavelength range from 790 to 815 nm is applied, and the ratio of the transmission and/or scattering power of these two wavelengths is calculated, with this ratio relative to the blood temperature serving as an indicator value for reading the blood glucose concentration from a calibration table. In the described method, the second wavelength is used to compensate for the influence of the glucose concentration on the scattering coefficients. A reliable temperature measurement is essential for the determination of the glucose concentration in this way. Outside the human body, this can be achieved by direct temperature measurement. Accordingly, the method described in DE 10 2006 036 920 can be used for example to monitor the blood sugar level for dialysis, since it is possible in that case to obtain an exact determination of the temperature of the blood outside of the body. The implementation of the method described in DE 10 2006 036 920 for the noninvasive in vivo determination of the glucose concentration also presupposes a noninvasive measurement of the blood temperature.

Such a method of the noninvasive, optical determination of the temperature of a medium within a body is known from DE 10 2008 006 245 [U.S. Pat. No. 8,426,819]. The medium to be examined is irradiated with infrared and/or visible light in the range of an absorption line whose position depends on the temperature of the medium, with the absorption of the light near the absorption line being measured and the temperature being determined from this measurement by comparison with calibration data. It is essential in this regard that the medium be irradiated with at least two discrete light wavelengths that lie near the absorption line on different sides of the absorption peak. At least one temperature-dependent measured value or temperature-dependent measurement function is determined from the relationship and/or a functional correlation of these two identified absorption values relative to one another, with the temperature being determined from this measured value and/or this measurement function by comparison with the previously recorded calibration data. During this optical temperature measurement as well, the location of the measurement inside a body, for example a blood vessel, can be marked by pulsed ultrasonic radiation. The primary focus here is a temperature measurement that is as exact as possible, which means that a temperature calibration is necessary in particular.

The described methods already enable the blood glucose concentration in flowing blood inside a body to be determined in vivo. The ultrasound localization described in EP 1 601 285 plays a special role in this regard. By a combination with the method that is known from DE 10 2008 006 245, the temperature of the blood can also be determined in vivo and taken into account in this analysis. However, these known methods could benefit from further development in order to optimize the quality of the measurement as well as the handling of a corresponding apparatus. This is where the invention comes in.

It is the object of the invention to provide a method that enables a simplified and improved noninvasive optical in vivo determination of the glucose concentration to be made in flowing blood in a blood vessel inside a body.

To attain this object, the invention teaches a method of the noninvasive in vivo determination of the glucose concentration in flowing blood in a blood vessel inside a body, wherein
the body is irradiated with (preferably pulsed) ultrasonic radiation at an ultrasonic frequency f_{US} for the purpose of marking a blood vessel,
the body with the blood vessel is irradiated with light having at least a first light wavelength at which the intensity of the backscattered light is dependent on the glucose concentration,
the body with the blood vessel is irradiated for the purpose of temperature compensation with at least a second light wavelength that lies near a water absorption line whose position is dependent on the temperature of the blood,

the backscattered light is detected using at least one sensor,
the signal components modulated at a modulation frequency that is dependent on the ultrasonic frequency are extracted using an evaluation unit from the sensor signals measured by the sensor,
an indicator value for the glucose concentration is determined from the signal component identified at the first wavelength, and
the indicator value is corrected with the signal component of the second light wavelength in order to compensate for temperature dependence.

First of all, the invention proceeds in this regard from the inherently known insight that characteristics of flowing blood, such as for example the glucose concentration, can be measured inside a body noninvasively and in vivo using optical methods if the point of measurement is simultaneously marked by ultrasonic radiation. The invention draws from known for example methods in this regard, EP 1 601 285 or DE 10 2008 006 245 or DE 10 2006 036 902 and further develops them. The primary focus is placed on determining the blood glucose concentration with at least one NIR wavelength from a range from 600 nm to 2500 nm. At least one wavelength is selected from this range at which the intensity of the backscattered light is dependent on the glucose concentration. This dependence can be dominated by interaction of the radiation as a result of scattering and/or absorption. For instance, it lies within the scope of the invention to select a first light wavelength near a glucose absorption line or glucose absorption band, for example from a range from 1560 to 1630 nm, preferably 1600 nm. In so doing, one can draw upon the insights from DE 10 2006 036 920. However, it was recognized according to the invention that, in order to determine the glucose concentration, not only wavelengths from near the absorption bands of glucose can be considered; instead, the glucose content also influences the (dynamic) scattering of light as a result for example of the contrast in refractive index between the tissue components, so wavelengths outside of glucose absorption bands can also be used. Moreover, it is advantageous if the first wavelength is selected from a range in which the intensity of the backscattered light is not dependent or is not substantially dependent on the oxygenation of the blood. Given this, a first wavelength can be preferably selected from a range from 790 to 815 nm, preferably 800 to 810 nm. After all, the two absorption curves of oxyhemoglobin and deoxyhemoglobin intersect in this range, so that backscattering that is dependent on the glucose concentration is independent of the oxygen content of the blood.

Alternatively, however, other wavelength ranges also merit consideration in which the backscattering is dependent on the glucose concentration but is not dependent or only marginally dependent on the oxygen load of the blood. The backscattering in the previously mentioned range around 1600 nm is also for example nearly independent of the oxygen load.

Consequently, in the context of a first preferred alternative, the first light wavelength can be selected from a range from 790 to 815 nm, preferably 800 to 810 nm.

In a second alternative, the first light wavelength can be selected from a range from 1500 to 1850 nm, for example 1550 to 1800 nm, preferably 1550 to 1750 nm.

Finally, as a third alternative, a range from 1000 to 1400, for example 1100 to 1400, preferably 1180 to 1250 nm can also be considered for the first wavelength.

In addition, the irradiation and detection is performed at a second and preferably a third wavelength which lie near a water absorption line and with which the temperature dependence can be compensated for. This will be discussed below in greater detail.

In addition to the first wavelength that is used, the backscattering of which is dependent on the glucose concentration of the blood, an additional (fourth) light wavelength is preferably used that is different from the first light wavelength and at which the intensity of the backscattered light is also dependent on the glucose concentration, however.

For example, if the first wavelength is selected from the range from 790 to 815 nm, then a wavelength from the range from 1000 to 1400, for example 1100 to 1400, preferably 1180 to 1250 nm, can be used as the fourth wavelength, for example. Alternatively, a fourth wavelength from the range from 1500 to 1850 nm, for example 1050 to 1800 nm, preferably 1550 to 1750 nm, and especially preferably a fourth wavelength near a glucose absorption line or glucose absorption band can be used.

All things considered, in selecting the fourth wavelength, the same ranges can be used that were already cited for the first wavelength, provided that different wavelengths are selected for the first wavelength and the fourth wavelength; especially preferably, wavelengths are selected from wavelength ranges that are different from those named in order to eliminate the various influences in the described manner.

The expressions “first” wavelength on the one hand and “fourth” wavelength on the other hand refer to a case in which two wavelengths (second and third wavelengths) are being used for temperature compensation. It will readily be understood that, in a case in which only one wavelength (second wavelength) is used for the temperature compensation, the described “fourth” wavelength is then used as the “third” wavelength. Moreover, it lies within the scope of the invention to select not only the first and optionally fourth wavelength for the determination of the glucose concentration, but also additional wavelengths from the described ranges (for example a fifth wavelength) as needed in order to further optimize the measurement.

Overall, the possibility exists according to the invention of using a plurality of wavelengths from different ranges in which the different influence quantities (for example hematocrit level, oxygenation, temperature, hemoglobin, glycated hemoglobin, etc.) of different sizes, so that the effects determined by the glucose can be extracted in a maximally targeted manner by appropriately combining at least two wavelengths. When using a first wavelength and a fourth wavelength and, optionally, a fifth wavelength, it is advantageous to be able to compare the identified signal components in order to determine an indicator value for the glucose concentration independently of other influences.

The above-described determination of the blood glucose concentration with the aid of at least the first wavelength and, optionally, the fourth (or even fifth) wavelength is combined with ultrasonic marking such as that known for example from EP 1 601 285. According to the invention, however, it is of particular importance that, in the process of determining the blood glucose concentration, the temperature dependence be compensated for directly without absolute temperature measurement and thus also without temperature calibration during measurement. According to the invention, the insights regarding temperature measurement in flowing blood that are known from DE 10 2008 006 245 are thus employed, but without a temperature calibration and hence an absolute temperature measurement. Instead, the measurement of the absorption near the temperature-dependent water absorption line is integrated directly into the measurement, such that the temperature dependence is compensated for directly from the interaction of the absorption/scattering of the glucose and the temperature-dependent water absorption.

The method according to the invention is therefore characterized by the combination of a plurality of measurements with at least two, preferably at least three light wavelengths and therefore for example two or three laser-light sources, with ultrasound localization occurring for all wavelengths thus enabling the signal components originating from the bloodstream can be extracted.

For temperature compensation, it can be sufficient to only apply and evaluate the second wavelength in the vicinity of a water absorption line. Especially preferably, however, the body or the blood vessel is irradiated with a third light wavelength that lies near the same water absorption line in order to compensate for the temperature influences, in which case the second and the third light wavelength lie on different sides of the absorption peak and the compensation of the temperature dependence is performed using the relationship of these two identified signal components to one another. In this case, the method of measuring temperature that is described in DE 10 2008 006 245 is employed, but without the corresponding temperature calibration, but rather directly during the measurement simply in order to compensate for the temperature dependence. In this preferred embodiment, at least three light wavelengths are therefore used, with the first light wavelength relating to the glucose dependence, and with the second light wavelength and the third light wavelength being used for the temperature compensation.

The second light wavelength and/or the third light wavelength are preferably 600 to 2500 nm, more preferably 800 nm to 1600 nm, for example 950 nm to 1000 nm. Tests have shown that measuring the temperature with the aid of infrared light near the water absorption band around 970 nm leads to outstanding results. In that case, at least one wavelength between 950 and 970 nm, for example, and for example at least one wavelength between 975 and 1000 nm are then used. It is also possible, however, to work with other water absorption bands within the biological window, for example near the water absorption band around 1450 nm. As a matter of principle, any absorption line can be considered whose position (wavelength of the peak) is temperature-dependent. If two light wavelengths near a water absorption line are used according to DE 10 2008 006 245, the medium is irradiated, as described in DE 10 2008 006 245, with two discrete light wavelengths that lie near the absorption line on different sides of the absorption peak, in which case a temperature-dependent correction value is determined from the ratio or a functional correlation of these two detected absorption values that is then used directly in the temperature compensation. This correction can be determined, for example, by finding the difference between the two absorption values lying on either side of the peak or by determining the slope of a line that runs through the measurement points.

The use of a plurality of light wavelengths and the detection thereof are the primary focus of the measurement. It lies within the scope of the invention for the measurements to be performed successively in time at the individual wavelengths, which makes it readily possible to work with the same sensor. Alternatively, however, it also lies within the scope of the invention to do the measurement simultaneously at the different wavelengths. One and the same sensor is preferably also used for this purpose. To enable the signals to be differentiated, it would be possible, for example, to modulate the individual lasers with different frequencies, so that the individual signals could be separated from one another by demodulation of the sensor signal. For simultaneously recording several wavelengths, the alternative possibility exists of using a plurality of for example three or four sensors that perform selective narrowband detection in respective wavelength ranges, for example also with the use of special filters or the like in front of the sensor surface.

In the context of ultrasound localization, the method known from EP 1 601 285 can be particularly used in which focused ultrasonic radiation is used. The ultrasonic radiation is focused inside a central blood vessel and a fixed pulse length and repetition time for the ultrasonic radiation is predetermined. During the analysis, the entire light component that is modulated at the frequency of the ultrasonic radiation is extracted, particularly independently of whether the light was in fact backscattered from the bloodstream or possibly from adjoining tissue. This is possible in this approach because the ultrasonic radiation is focused on the bloodstream, so that the modulated component of the light that is backscattered outside of the bloodstream is small.

Alternatively, however, a further developed method can be used for the ultrasound localization in which it is possible to use unfocused ultrasonic radiation. In that case, the body is irradiated at an ultrasonic frequency f_{US} in order to mark a blood vessel with ultrasonic radiation and at the same time the blood vessel is irradiated in the inherently known manner with light at the desired light wavelength and the backscattered light is detected with the sensor. The light component backscattered from the body outside of the blood vessel is modulated at a frequency f_{MG}, which corresponds to the frequency f_{US} of the ultrasonic radiation. In contrast, due to the Doppler effect in flowing blood, the light component that is backscattered within the blood vessel is modulated at a frequency f_{MB}, which is shifted by the Doppler shift f_{D} relative to the frequency f_{US} of the ultrasonic radiation. Using the evaluation unit, the signal component that is modulated at the shifted frequency f_{MB} is then extracted from the sensor signal measured by the sensor. This optimized variant ensures that only those light components of the backscattered light are used in the analysis that are in fact backscattered from the blood. In this respect, the invention proceeds from the insight that the backscattered light components from the flowing blood on the one hand and from the surrounding tissue on the other hand are modulated at different modulation frequencies. In the surrounding tissue, the modulation frequency is equal to the ultrasonic frequency. In the flowing blood, however, a modulation occurs at a different frequency due to the Doppler effect. According to the invention, it is thus possible to precisely locate the bloodstream by exploiting the Doppler effect independently in fact of whether or not focused ultrasonic radiation is used. In that case, it is advantageous if the body is irradiated with pulsed ultrasonic radiation with a predefined pulse length and repetition time, with the light intensity at the sensor being measured in a time window that is time-shifted by a delay that corresponds to the pulse length of the ultrasonic radiation. In this method as well, the blood vessel is first located in principle before the optical measurement by an acoustic analysis of the ultrasonic echo backscattered from the body.

As mentioned above, the method according to the invention with ultrasound localization is characterized in that only intensities and thus only photon flux is measured with the sensor. Nonetheless, it will readily be understood that the signal components are extracted during the analysis that are modulated at the respectively relevant frequencies as a function of the ultrasonic radiation. Standard methods for isolating low frequencies from high-frequency mixed signals can be used, such as radar signal analysis.

The object of the invention is also an apparatus for the noninvasive optical in vivo determination of the glucose concentration in flowing blood in a blood vessel inside a body according to a method of the described type, comprising at least
one ultrasound source,
one first laser light source for generating the first light wavelength,
one second laser light source for generating the second light wavelength,
optionally, one third laser light source for generating the third light wavelength,
one optical sensor for detecting the backscattered light,
one control and evaluation unit that is connected to the ultrasound source, the laser light sources, and the sensor,
   wherein
the respective signal components modulated at a modulation frequency that is dependent on the ultrasonic frequency can be extracted using the control and evaluation unit from the sensor signals measured by the sensor,
an indicator value for the glucose concentration is determined from the signal component identified at the first wavelength, and
the indicator value is corrected with the signal components of the second light wavelength in order to compensate for the temperature dependence.

Preferably, the apparatus also has a third laser light source for generating the third light wavelength, so that two light wavelengths near a water absorption line are then available for the temperature compensation. Optionally, a fourth laser light source is also used which makes an additional “glucose-dependent” wavelength available.

An ultrasound source that emits a strongly directional ultrasonic beam is therefore part of the apparatus. Depending on which method is used for ultrasound localization, focused or unfocused ultrasonic radiation can be used. A homogeneous ultrasonic pressure on an order of magnitude of about 1 MPa is generated in the lateral and axial direction. Especially preferably, the angle of incidence of the ultrasonic radiation can be varied with the ultrasound emitter, particularly electronically but also mechanically as needed. Adjusting the angle facilitates the search of the blood vessel at different depths in order to enable the point of interaction between light and ultrasound to be maintained at the same location relative to the sensor. The ultrasound frequency used depends for example on what type of blood vessel is being examined; if measurement is being performed near the radial artery, an ultrasonic frequency of 3.8 MHZ can for example be advantageous. Pulsed ultrasonic radiation is preferably used, in which case the pulse repetition rate is dependent on the depth of the location being examined. During the actual measurement, ultrasonic radiation is produced that modulates the optical signal at the optical sensor. Prior to the measurement, however, it is necessary to record the ultrasound echo in order to locate the bloodstream, so the ultrasound emitter should have a transducer that enables the ultrasonic radiation to not only be generated but also detected.

According to the invention, the light sources are preferably laser light sources, with at least three, but preferably four laser light sources being used. The laser light sources generate continuous, monochromatic, and coherent light of the respective wavelength. It lies within the scope of the invention for the laser be switched, that is turned on or off, individually by the controller according to the measurement algorithm. It lies within the scope of the invention to perform the measurements in succession, so that a simple evaluation is performed in a single sensor. Alternatively, the measurements can also be performed at the same time, in which case the individual laser beams are modulated at respective different frequencies, so that the sensor signal can be demodulated appropriately in order enable a determination to be made as to which signal component corresponds to which incident light radiation even when measuring simultaneously.

In order to provide an apparatus for determining blood glucose concentration that is compact overall, it is proposed that all of the lasers be arranged on a support in the center of a sensor head, but that a laser emitter be provided with a plurality of lasers. When switching on the lasers one after the other, it would be possible to use a driver, a cooling system, and a power supply for all of the lasers.

Different sensors can be used according to the invention. The sensors themselves are preferably formed by diodes. It lies for example within the scope of the invention for a sensor to be used having an annular measuring surface, in which case the point of incidence of the laser light is at the center of this annular measuring surface. One can draw in this regard upon the sensor array known from DE 10 2007 020 078. The reasoning behind this is that the deeper the backscattered photons are scattered in the tissue, the farther away from the point of incidence they will exit the tissue. Statistically speaking, in investigations of tissue at a very specific depth, the scattered light with a particularly high intensity is detected at a certain spacing from the point of incidence, which corresponds to approximately half the depth of the scattering center. This circumstance is exploited for example by providing an annular measuring surface around the point of incidence; the diameter of the annular surface that is being measured preferably corresponds approximately to the depth of the area to be studied. However, the fact that the intensity is dependent on the spacing from the irradiation point as a function of the depth of the scattering center can also be exploited in other sensor designs.

Moreover, by incorporating appropriate localization using ultrasound, the design of the sensor can take into account the fact that the intensities of the photon flux are random variables with statistical properties. When coherent light “diffuses” through a medium, the emitted light has a “speckled pattern,” with so-called “speckles.” A speckle is a point of light in which the signal is coherent. The average area A of a typical speckle is approximately A=α·λ², where λ is the wavelength of the light and α, in the present case, is between 3 and 5. Detection can be optimized if the smallest possible number of speckles reaches the sensor in an observed moment. It would therefore be especially advantageous for a plurality of small sensors to be read out simultaneously. With appropriate time and effort, this could be achieved using multichannel electronics. In order to keep the structure of the sensor simple and thus the costs low, it is proposed in a preferred development that the sensor be embodied as a diode array or linear array with a plurality of diodes that, when seen from above, are next to one another so as to be transverse or perpendicular to the direction of the ultrasonic radiation. The individual signals are for example added. For instance, the ultrasonic radiation is radiated obliquely into the body at a predefined angle of incidence, and the sensor is on the opposite side with respect to the point of entry of the light into the body. This configuration has the advantage that the backscattered speckles are not repeatedly modulated by the ultrasonic radiation, so that the measurement result is improved. As an alternative to a diode array or a linear array, it is also possible to use a single sensor or individual sensor or a corresponding diode; it can for example be rectangular, and its long axis can extend transverse to the direction of propagation of the ultrasonic radiation and/or transverse to the light path.

The invention is explained in further detail below with reference to a schematic drawing, which illustrates only one embodiment.

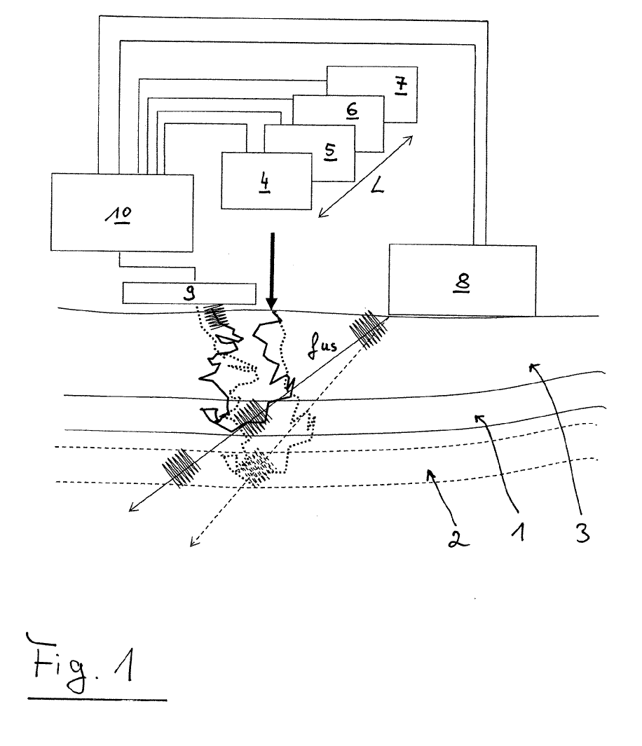

FIG. 1 is a simplified schematic view of an apparatus according to the invention,

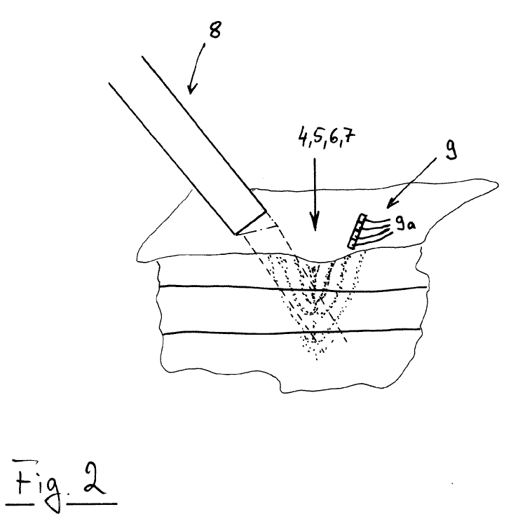

FIG. 2 is a simplified view of the assembly of the ultrasonic source and the sensor, and

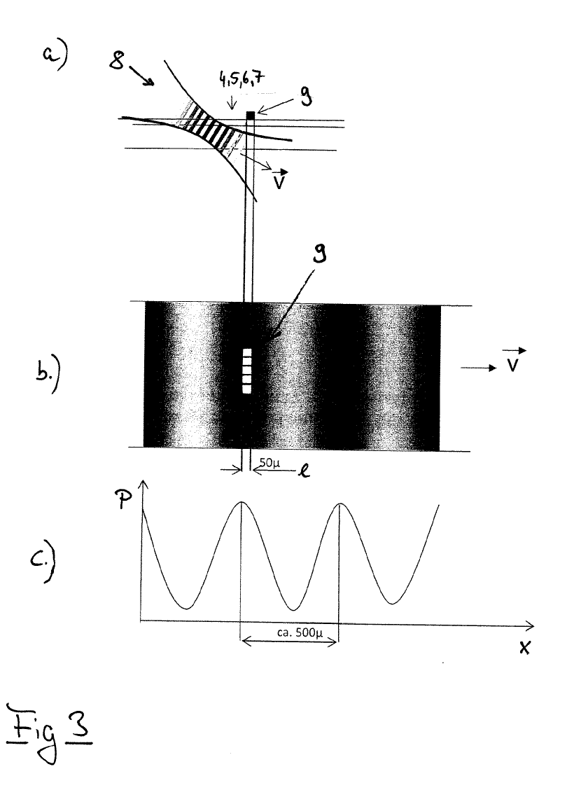

FIG. 3 is another view of the situation according to FIG. 2.

FIG. 1 shows a body with two blood vessels 1 and 2 and the tissue 3 adjoining the blood vessels 1 and 2. In order to perform noninvasive optical measurement of the glucose concentration in the flowing blood, a laser emitter L with a plurality of lasers 4, 5, 6, 7, an ultrasound emitter 8, a sensor 9, and a control and evaluation unit 10 are provided. The body with the blood vessel 1 is irradiated with laser light from the lasers 4, 5, 6, 7. The backscattered light is detected by the sensor 9. This sensor 9 measures only intensities; that is the backscattered photon flux is detected without spatial resolution or (optical) frequency resolution at the sensor.

According to the invention, the body is irradiated with ultrasonic radiation at a defined ultrasonic frequency f_{US} for the purpose of marking the blood vessel 1. Due to the interaction of the ultrasonic radiation with the blood and/or tissue, the backscattered light components are modulated at the frequency of the ultrasonic radiation. In this way, the signal components modulated at a modulation frequency that is dependent on the ultrasonic frequency f_{US} can be extracted using the evaluation unit 10 from the sensor signals measured by the sensor 9.

According to the invention, a plurality of lasers 4, 5, 6, 7 with different light wavelengths are used to determine the blood glucose concentration. The first laser 4 has a light wavelength at which the backscattered signal component is dependent on the glucose concentration and consequently represents the glucose concentration, thus enabling a corresponding indicator value to be determined. This can be a light wavelength in the range between 790 nm and 850 nm, for example about 805 to 808 nm; after all, the two absorption curves of oxyhemoglobin and deoxyhemoglobin intersect in this range, so that, at this wavelength, the absorption and thus the proportion of backscattered light is independent of the state of oxygenation.

An additional laser, referred to in this case as the fourth laser 5, can also be used to emit another light wavelength at which the backscattering is dependent on the glucose concentration. This fourth laser can have a wavelength in a range from 1180 nm to 1250 nm; alternatively, it can also have a wavelength in a range from 1550 nm to 1750 nm.

Of particular importance is the fact that the additional lasers 6, 7, namely the second laser 6 and the optional third laser 7, are used for temperature compensation. The second light wavelength can be 950 to 970 nm, for example, and the third light wavelength can for example be 975 nm to 1000 nm, so that these two light wavelengths lie on different sides of the absorption peak of a water absorption line at for example 970 nm. The ratio of the two signal components originating from the backscatter of the second light wavelength and the third light wavelength has a very sensitive dependence on the temperature of the medium, which means that these values can then be used directly for compensation of the temperature dependence. It is crucial in this regard that no absolute temperature measurement is required and therefore no previous temperature calibration is provided. It is perfectly sufficient to also register the second and third wavelengths while determining the glucose concentration by utilizing the first wavelength (and, optionally, the fourth wavelength) and to correct the measured values accordingly.

Moreover, FIG. 1 shows that two blood vessels 1 and 2 lie one over the other. Nevertheless, a perfect localization and separation of the signals is possible in the context of the invention. First, this is due to the fact that the scattered light, as described for example in DE 10 2007 020 078, occurs, statistically speaking, with an especially high intensity at a certain spacing from the point of incidence (of the light), particularly at a spacing from the point of incidence that corresponds to approximately half the depth of the scattering center. Given a certain geometry, the signal that may have been backscattered from another layer on the correspondingly positioned sensor is therefore substantially weaker than the relevant signal. FIG. 1 shows this using the example of the two situations that are illustrated. The upper blood vessel 1 is to be evaluated. If the angle of incidence of the ultrasonic radiation is varied with the aid of a variable angle setting of the ultrasound source 8, then backscattered photons that are modulated in the area of the lower bloodstream 2 can also strike the sensor 9. This situation (indicated by dashed lines) then results however in significantly lower intensities. The method is optimized by working with pulsed ultrasonic radiation, with the ultrasonic radiation having a predefined pulse length and repetition time, and with the light intensity at the sensor 9 then being measured in a time window that is time-shifted by a delay that corresponds to the pulse length of the ultrasonic radiation. This can also ensure that only the photons that are backscattered from the area of the upper blood vessel are actually detected. It is important to note that the sensor is substantially over-dimensioned in FIG. 1. For a selection between the signals of bloodstreams that are lying one over the other, it is advantageous to work with only a very small sensor, since, owing to the afore-described statistical dependence, it is then ensured that the light is backscattered from a certain depth with an especially high intensity. Preferably, the sensor should have a size that is on the scale of a speckle. The average area of a speckle is approximately A=α·λ², where λ is the wavelength of the light and α here is between 3 and 5.

FIG. 2 gives a schematic indication of one preferred arrangement of the individual components during measurement. It can be seen that the ultrasonic radiation is preferably radiated obliquely into the body at a predefined angle and that the sensor 9 is arranged relative to the incident light of the opposite side. In the embodiment shown there, a diode array with a plurality of individual diodes 9a is provided as the sensor 9, with the outputs being summed. By virtue of the illustrated situation, backscattered photons are prevented from also being modulated multiple times by the ultrasonic radiation on the way back through the tissue. This enables the signal-to-noise ratio to be improved.

The situation is also elucidated in FIG. 3. There, the situation according to FIG. 2 is shown in a side view in the upper area a) and in a top view in the middle area b). The middle area b) graphically shows the variation in the ultrasonic pressure. Accordingly, the lower area c) of FIG. 3 shows the ultrasonic pressure P as a function of the location x. FIGS. 3b) and 3c) are different views of the ultrasonic pressure P as a function of the path or length X. The velocity vector V of the ultrasonic wave is indicated. It can be seen that the sensor 9 has a very short length l in the direction of propagation of the ultrasonic wave, which here is 50 μm. It can lie for example between 10 μm and 100 μm. The primary aim of this dimensional design is to ensure that the smallest possible number of speckles reaches the sensor 9 in a given moment. Since the signal becomes weak due to such small dimensioning and consequently has a small length l, a plurality of sensors 9a are arranged next to one another, such that the signal can be strengthened. In addition, the short length l of the sensor has the advantage that, due to the statistical effects described above, a better separation between signals that might result from superposed bloodstreams is made possible. A small extension on the part of the sensor is therefore advantageous. What is meant here that the sensor does not extend far in the direction defined by the spacing between the point of incidence of the laser radiation and the sensor. This direction may also correspond to the direction of propagation of the ultrasonic wave. Reference is made in this regard to the figures. As an alternative to a sensor with individual sensors, it is also possible to use an individual sensor (having a rectangular shape, for example), which can also be dimensioned and arranged so as to have a short length and a greater width.

Even if, as described, no temperature calibration is required according to the invention because, in the absence of an absolute temperature measurement, only a temperature-dependent compensation is performed, calibration of the system is required beforehand in order to determine the glucose concentration. For this purpose, various glucose situations can be carried out with the system using a gold standard reference system in vivo on suitable subjects. Since the information about the hematocrit value can also be provided by optical measurement, it is also possible to obtain the correct measurement using full blood or plasma calibration.

The actual measuring process can then be carried out without further calibration. The sensor can be placed on the body, such as on the forearm, directly over a blood vessel such as for example the radial artery. It should be noted that a location should be chosen where the blood vessel is not too deep, with an ideal depth being less than 1 cm. The artery is first searched for using known methods on the ultrasound system. This can be done acoustically by evaluating the ultrasonic radiation that is reflected back. Once the search is completed, the optical measurement is started automatically. The optical measurement consists of an optimized sequence of light pulses of the respective wavelengths in order to scan all physiologically altered scattering and absorption situations over the course of several heartbeats. The signals undergo analog/digital analysis and are stored in raw data tables/arrays. This is followed by an evaluation of the indicator values determined in this way and a comparison with corresponding calibration data for determining the blood glucose concentration.

1. A method of the noninvasive in vivo determination of the glucose concentration in flowing blood in a blood vessel inside a body, the method comprising the steps of:
irradiating the body with ultrasonic radiation at an ultrasonic frequency for marking the blood vessel;
irradiating the body with the marked blood vessel with light having at least a first light wavelength at which the intensity of backscattered light is dependent on glucose concentration;
irradiating the body with the marked blood vessel with light having a second light wavelength that lies in a water absorption line whose position is dependent on the temperature of the blood;
detecting backscattered light using a sensor;
extracting respective signal components modulated at a modulation frequency dependent on ultrasonic frequency using an evaluation unit from signals outputted by the sensor
determining an indicator value for glucose concentration is determined from a signal component identified at the first wavelength; and
correcting the indicator value with a signal component of the second light wavelength to compensate for temperature dependence.

2. The method defined in claim 1, further comprising the step of:
irradiating the body or the blood vessel with a third light wavelength that lies in the water absorption line in order to compensate for temperature influences, the second and third light wavelength lying on different sides of the absorption peak, the compensation of the temperature dependence being performed using the relationship of these two identified signal components to one another.

3. The method defined in claim 2, further comprising the step of:
irradiating the body with the blood vessel with a fourth light wavelength that is different from the first light wavelength and at which the intensity of the backscattered light is also dependent on glucose concentration.

4. The method defined in claim 1, wherein light wavelengths in a range from 600 nm to 2500 nm are used.

5. The method defined in claim 3, wherein the first or the fourth light wavelength are selected from a range in which the intensity of the backscattered light is not dependent or is not substantially dependent on oxygenation of the blood.

6. The method defined in claim 3, wherein the first or the fourth light wavelength is selected from a range from 790 nm to 815 nm.

7. The method defined in claim 3, wherein the first light wavelength or the fourth light wavelength is selected from a range from 1000 nm to 1400 nm.

8. The method defined in claim 3, wherein the first light wavelength or the fourth light wavelength is selected from a range from 1500 nm to 1850 nm.

9. The method defined in claim 2, wherein the second light wavelength or the third light wavelength is 600 nm to 2500 nm.

10. The method defined in claim 9, wherein the second light wavelength is 950 to 970 nm and the third light wavelength is 975 to 1000 nm.

11. The method defined in claim 1, wherein the steps of irradiating are performed successively in time at different wavelengths.

12. The method defined in claim 1, wherein the steps of irradiating are performed simultaneously at respective different wavelengths.

13. The method defined in claim 1, further comprising the step of:
extracting light components modulated at ultrasonic frequency with the evaluation unit from respective sensor signals, the ultrasonic radiation being focused on the bloodstream.

14. The method defined in claim 1, further comprising the steps of:
modulating light components backscattered from the body outside the blood vessel at a frequency that corresponds to a frequency of the ultrasonic radiation;
modulating a light component backscattered within the blood vessel at a frequency that is shifted by Doppler shift relative to the frequency of the ultrasonic radiation due to the Doppler effect in flowing blood; and
extracting signal components modulated at the shifted frequency using the evaluation unit from the sensor signals measured by the sensor.

15. An apparatus for the noninvasive optical in vivo determination of the glucose concentration in flowing blood in a blood vessel inside a body, the apparatus comprising:
an ultrasound source;
a first laser light source for generating a first light wavelength;
a second laser light source for generating a second light wavelength;
an optical sensor for detecting backscattered light;
a control and evaluation unit connected to the ultrasound source, the laser light sources, and the sensor, and
evaluating means for:
extracting the respective signal components modulated at a modulation frequency that is dependent on the ultrasonic frequency from the sensor signals measured by the sensor,
determining an indicator value for the glucose concentration from a signal component identified at the first wavelength, and
correcting the indicator value with a signal component of the second light wavelength in order to compensate for the temperature dependence.

16. The apparatus defined in claim 15, further comprising:
a third and fourth laser light sources for generating third and fourth light wavelengths.

17. The apparatus defined in claim 15, further comprising:
means for varying an angle of incidence of the ultrasonic radiation with an ultrasonic emitter.

18. The apparatus defined in claim 15, wherein the sensor is a diode array with a plurality of diodes that, when seen in a view of the body from above, are next to one another so as to be transverse to a direction of propagation of the ultrasonic radiation and/or transverse to the light path, or that the sensor is a rectangular single sensor that is oriented with its longitudinal axis transverse to the direction of propagation of the ultrasonic radiation and/or transverse to the light path.
